(19) United States
(12) Patent Application Publication
(10) Pub. No.: US 2009/0088669 A1
(43) Pub. Date: Apr. 2, 2009

(54) Vacuum Disruption Apparatus With Triple Variable Intersecting Ultrasonic Beams

(75) Inventor: Kokichi Ido, Chiba-shi (JP)

Correspondence Address: RANKIN, HILL & CLARK LLP, 925 EUCLID AVENUE, SUITE 700, CLEVELAND, OH, 44115-1405, US

(73) Assignee: ELEKON SCIENCE CO., LTD., Chiba-shi (JP)

(21) Appl. No.: 12/243,071

(22) Filed: Oct. 1, 2008

(30) Foreign Application Priority Data
Oct. 2, 2007 (JP) 2007-258356

(51) Int. Cl. A61N 7/00

(52) U.S. Cl. 601/2

(57) ABSTRACT

A vacuum disruption apparatus with triple variable intersecting ultrasonic beams, capable of disrupting a great number of cells efficiently without chemical loading. The apparatus includes a vacuum disruption vessel for containing and sealing a sample solution having living cells to be disrupted; at least three ultrasonic generation units for emitting ultrasonic beams toward the vacuum disruption vessel; and an ultrasonic modulation unit for varying the intensities and frequencies of ultrasonic from the ultrasonic generation units.

### RELATED APPLICATIONS

This application claims the priority of Japanese Patent Application No. 2007-258356 filed on Oct. 2, 2007, which are incorporated herein by reference.

### FIELD OF THE INVENTION

The present invention relates to disruption apparatuses, and more specifically, to a vacuum disruption apparatus with triple variable intersecting ultrasonic beams, for disrupting cells.

### BACKGROUND OF THE INVENTION

Extraction of intracellular components requires cell disruption. In mass disruption of living cells, prolonged overloading of cells must be avoided.

Cells, especially cells of animals, without cell walls have a low physical strength and a high resilience, and mass and quantitative disruption of those cells require advanced skills.

Conventionally, an ultrasonic beam has been applied to disrupt relatively small objects, such as cells, under moderate conditions. For example, an apparatus disclosed in Japanese Unexamined Patent Application Publication No. 2005-211837 has been used, but the disruption has been often uneven and has not necessarily been satisfactory in terms of disruption rate.

Injection of a substance into cells has also been conducted to disrupt cells. If the substance to be transferred into a cell is a low-molecular substance, the cell may absorb the substance, but high-molecular substances such as protein and genes require forced transfer, which requires advanced manual operation skills such as injection into each cell by a capillary tube.

### DISCLOSURE OF THE INVENTION

### Problem to be Solved by the Invention

In view of the related art described above, it is an object of the present invention to provide an apparatus for disrupting a relatively small object efficiently without chemical loading.

### Means to Solve the Problem

A vacuum disruption apparatus with triple variable intersecting ultrasonic beams, according to the present invention includes

a vacuum disruption vessel for containing and sealing a sample to be disrupted;

at least three ultrasonic generation units for emitting ultrasonic beams toward the vacuum disruption vessel; and

an ultrasonic modulation unit for varying intensities and frequencies of the ultrasonic from the ultrasonic generation units.

It is preferred that the vacuum disruption vessel of the apparatus has a vacuum pressure of 1 to 10⁻³ Pa.

It is preferred that the ultrasonic modulation unit of the apparatus can vary the oscillating frequency of ultrasonic from each of the ultrasonic generation units within a range of 17 to 20 kHz.

It is preferred that the disruption apparatus disrupts cells.

It is preferred that the apparatus transfers a high-molecular substance included in a dispersion medium for cell into cells.

The disruption apparatus according the present invention emits three intersecting ultrasonic beams toward the object to be disrupted in a vacuum, so that highly-efficient and uniform disruption can be performed.

If the object to be disrupted is a living cell, a high-molecular substance, such as a gene or protein, can be introduced into the cell efficiently by disrupting just a part of the cell membrane and allowing the high-molecular substance to coexist in the dispersion medium for cell.

### BRIEF DESCRIPTION OF THE DRAWINGS

FIG. 1 is a sectional side view of a vacuum disruption apparatus with triple variable intersecting ultrasonic beams, of an embodiment of the present invention.

FIG. 2 is a top view of the apparatus shown in FIG. 1.

### DESCRIPTION OF THE PREFERRED EMBODIMENT

A preferred embodiment of the present invention will be described below with reference to the drawings.

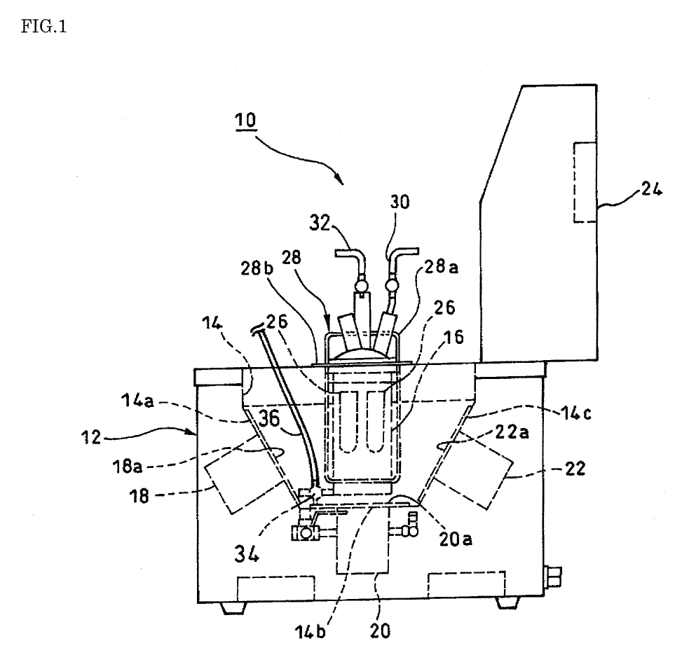

FIG. 1 is a side view showing a general structure of a vacuum disruption apparatus 10 with triple variable intersecting ultrasonic beams, of an embodiment of the present invention.

The disruption apparatus 10 shown in FIG. 1 includes a processing tank 14 disposed in an upper part of a main unit 12 of the apparatus, a vacuum disruption vessel 16 disposed in the processing tank 14, a first ultrasonic generator 18, a second ultrasonic generator 20, and a third ultrasonic generator 22 which are disposed at the bottom part of the processing tank 14, and an ultrasonic modulator 24 with an operating panel for varying the intensities and frequencies of ultrasonic from each of the ultrasonic generators 18, 20, and 22.

The processing tank 14 has an inverted trapezoidal bottom consisting of a first oblique face 14a on which an oscillating face 18a of the ultrasonic generator 18 is disposed, a horizontal bottom face 14b on which an oscillating face 20a of the ultrasonic generator 20 is disposed, and a second oblique face 14c on which an oscillating face 22a of the ultrasonic generator 22 is disposed.

In the processing tank 14, the vacuum disruption vessel 16 made of metal or glass, which propagates vibration (ultrasonic) well, is disposed. The ultrasonic generators 18, 20, and 22 are disposed in such a manner that the normal lines of the oscillating faces 18a, 20a, and 22a intersect in the vacuum disruption vessel 16. The processing tank 14 and the vacuum disruption vessel 16 contain water. In the vacuum disruption vessel 16, a plurality of glass containers 26, which transmit ultrasonic easily, can be placed.

In the ultrasonic modulator 24, the oscillating frequencies, oscillating intensities, and emission modes (continuous emission or intermittent emission) for each of the ultrasonic generators 18, 20, and 22, and the unit emission time, unit interval time, processing time, and the like for the intermittent emission mode can be changed with the operating panel. The temperature of the water in the processing tank 14, the vacuum pressure of the vacuum disruption vessel 16, and the like can also be changed on the same panel. It is also preferred that a computer which is separately connected to the apparatus can be used for changing the above-mentioned conditions instead of the operating panel.

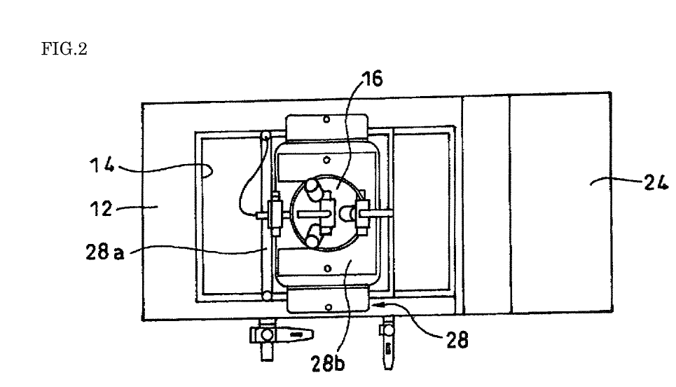

The vacuum disruption vessel 16 is held by a holder 28 and placed in the processing tank 14, and the vacuum disruption vessel 16 can be mounted and removed together with the holder 28. The structure of the holder 28 is as shown in the top view of the apparatus in FIG. 2. As clearly shown in the figure, the holder 28 has a handle 28a formed by a rectangular frame and a panel 28b for holding the top of the vacuum disruption vessel 16, both edges of the panel 28b being secured to the top of the main unit 12 of the apparatus.

In this embodiment, the vacuum disruption vessel 16 has a sample solution inlet valve 32 at the top and a sample solution outlet valve 34 at the bottom. The sample solution outlet valve 34 is connected to a peristaltic pump, which is not shown in the figure, through an outlet tube 36. The valves 32 and 34 allow continuous incoming and outgoing flow of a sample solution.

The vacuum disruption apparatus 10 with triple variable intersecting ultrasonic beams, of the embodiment is configured, in outline, as described above and the operation thereof will be described next.

Cell Disruption

A glass container 26 containing a cell suspension is placed in the vacuum disruption vessel 16, and then the vacuum disruption vessel 16 (having a capacity of about 3 liters) is sealed and placed in the processing tank 14 (having a capacity of about 12 liters). The vacuum disruption vessel 16 is decompressed by a vacuum pump, which is not shown in the figure, through a decompression valve 30. It is preferred to bring the vacuum pressure to 1 to 10⁻³ Pa, for disruption of usual cells of living creatures.

The ultrasonic generators 18, 20, and 22 start to generate ultrasonic oscillations with an operation in the ultrasonic modulator 24. The oscillating intensities and oscillating frequencies at the first, second, and third ultrasonic generators 18, 20, and 22 are as listed below.

TABLE 1

| | Oscillating frequency | Oscillating intensity |
|---|---|---|
| First ultrasonic | 20 kHz | 200 W |
| generator | | |
| Second ultrasonic | 20 kHz | 200 W |
| generator | | |
| Third ultrasonic | 20 kHz | 200 W |
| generator | | |

A unit time of ultrasonic emission was set to 10 seconds, and a unit time of emission interval was also set to 10 seconds. The ultrasonic emission and interval were repeated alternately for one hour.

The resultant cell disruption rate was about 100% (when observed with a microscope).

When the apparatus was used as a double-intersecting-ultrasonic-beam disruption unit by activating the first and second ultrasonic generators only, the disruption rate was about 40%. When the first, second, and third ultrasonic generators were activated in an atmosphere, the disruption rate was 50%.

Gene Injection

As in cell disruption, a glass container 26 containing a cell suspension is placed in the vacuum disruption vessel 16, and then the vacuum disruption vessel 16 is sealed and decompressed by the vacuum pump, which is not shown in the figure, through the decompression valve 30. It is preferred to bring the vacuum pressure to about 10⁻² Pa, for injection into usual cells of living creatures.

The ultrasonic generators 18, 20, and 22 start to generate ultrasonic oscillations with an operation in the ultrasonic modulator 24. The oscillating intensities and oscillating frequencies at the first, second, and third ultrasonic generators 18, 20, and 22 are as listed below.

TABLE 2

| | Oscillating frequency | Oscillating intensity |
|---|---|---|
| First ultrasonic generator | 19 kHz | 150 W |
| Second ultrasonic generator | 19 kHz | 150 W |
| Third ultrasonic generator | 19 kHz | 150 W |

A unit time of ultrasonic emission was set to 3 seconds, and a unit time of emission interval was set to 10 seconds. The ultrasonic emission and interval were repeated alternately for half an hour.

The resultant rate of gene transfer into cells was about 50%.

When the apparatus was used as a double-intersecting-ultrasonic-beam disruption unit by activating the first and second ultrasonic generators only, the transfer rate was 10% to 20%. When the first, second, and third ultrasonic generators were activated in an atmosphere, the transfer rate was also 10% to 20%.

Frequency and Transfer Rate

The inventors studied the relationship between the ultrasonic oscillating frequency and the gene transfer rate. The oscillating frequency was varied, and the other conditions were the same as those of the gene injection, described above.

TABLE 3

| | Oscillating frequency (kHz) | | |
|---|---|---|---|
| | 17 | 19 | 20 |
| Transfer rate | 40% | 50% | 45% |

When the apparatus according to the present invention is used for gene transfer into cells, a preferred oscillating frequency and oscillating intensity may depend on the type of the cell.

Because three ultrasonic beams are emitted to intersect with one another in the present invention, uniform disruption can be performed even in continuous processing of a sample solution.

1. A vacuum disruption apparatus with triple variable intersecting ultrasonic beams, comprising:
a vacuum disruption vessel for containing and sealing a sample to be disrupted;
at least three ultrasonic generation units for emitting ultrasonic beams toward the vacuum disruption vessel; and
ultrasonic modulation units for varying intensities and frequencies of ultrasonic from the ultrasonic generation units.

2. The vacuum disruption apparatus with triple variable intersecting ultrasonic beams of claim 1, wherein the vacuum disruption vessel has a vacuum pressure of 1 to 10⁻³ Pa.

3. The vacuum disruption apparatus with triple variable intersecting ultrasonic beams of claim 1, wherein the ultrasonic modulation units can vary the oscillating frequency of ultrasonic from each of the ultrasonic generation units within a range of 17 to 20 kHz.

4. The vacuum disruption apparatus with triple variable intersecting ultrasonic beams of claim 3, wherein the apparatus is used for disrupting cells.

5. The vacuum disruption apparatus with triple variable intersecting ultrasonic beams of claim 3, wherein the apparatus is used to transfer a high-molecular substance included in a dispersion medium for cell into cells.

6. The vacuum disruption apparatus with triple variable intersecting ultrasonic beams of claim 4, wherein the apparatus is used to transfer a high-molecular substance included in a dispersion medium for cell into cells.
